(19) United States
(12) Patent Application Publication
(10) Pub. No.: US 2021/0291159 A1
(43) Pub. Date: Sep. 23, 2021

(54) INTEGRATED SYSTEM FOR SAMPLING AND PROCESSING A LIQUID SUSPENSION

(71) Applicant: OKINAWA INSTITUTE OF SCIENCE AND TECHNOLOGY SCHOOL CORPORATION, Kunigami-gun Okinawa (JP)

(72) Inventors: Kazumi Toda-Peters, Onna-son (JP); Amy Shen Fried, Onna-son (JP); Shivani Sathish, Onna-son (JP); Doojin Lee, Uruma-shi (JP); Casey James Galvin, Yomitan-son (JP); Kei Funakoshi, Uruma-shi (JP)

(21) Appl. No.: 16/614,836

(22) PCT Filed: May 18, 2018
(86) PCT No.: PCT/JP2018/019248

§ 371 Date: Nov. 19, 2019

Related U.S. Application Data
(60) Provisional application No. 62/509,244, filed on May 22, 2017.

(51) Int. Cl. B01L 3/00; G01N 1/40; G01N 33/49

(52) U.S. Cl. CPC B01L 3/502; G01N 33/24; B01L 3/502715; G01N 1/4077; G01N 1/405; G01N 33/491; B01L 2200/0631; B01L 2200/027; B01L 2200/0621; B01L 2300/047; B01L 2300/0681; B01L 2300/087; B01L 2400/06; G01N 2001/4088; B01L 3/5635

(57) ABSTRACT

An integrated system for sampling and processing a liquid suspension, the system comprising: a cartridge member comprising a sampling port on a first end of the cartridge member, a second one-way valve in communication with the sampling port, a stopper at a second end of the cartridge member, and a fluid chamber between the second one-way valve and the stopper; a body member having a first end configured to accept the first end of the cartridge member and a second end opposite the first end of the body member; and a cap unit disposed on the second end of the body member, the cap unit comprising a wash chamber, wherein the cap unit comprises a first one-way valve between the second end of the body member and the wash chamber and a filter between the first one-way valve and the wash chamber or a filter between the second end of the body member and the wash chamber and a first one-way valve between the filter and the wash chamber.

### CROSS-REFERENCE TO RELATED APPLICATION

This application claims priority to U.S. Provisional Patent Application No. 62/509,244, filed on May 22, 2017, at the United States Patent and Trademark Office.

### TECHNICAL FIELD

Disclosed is an integrated system for sampling and processing a liquid suspension and a method of sampling and processing a liquid suspension using the integrated system.

### BACKGROUND ART

Many samples intended for analysis are liquid suspensions, such as whole blood or soil samples. Separating the liquid from the solid can be an important step in analyzing the liquid phase of the suspension. Existing techniques often require electricity and/or are expensive. Furthermore, often a portable system that does not use electrical power is desirable.

### SUMMARY OF INVENTION

Disclosed is an integrated system for sampling and processing a liquid suspension, the system including: a cartridge member comprising a sampling port on a first end, a one-way valve, a stopper, and a fluid chamber between the one-way valve and the stopper; a body member having a first end configured to accept a first end of the cartridge member and a second end opposite the first end of the body member; and a cap unit disposed on the second end of the body member, the cap unit comprising a wash chamber, wherein the cap unit comprises a one-way valve between the second end of the body member and the wash chamber and a filter between the one-way valve and the wash chamber, or a filter between the second end of the body member and the wash chamber and a one-way valve between the filter and the wash chamber.

Also disclosed is a method of sampling and processing a liquid suspension, the method comprising: providing a cartridge member comprising a sampling port on a first end, a one-way valve, a stopper, and a fluid chamber between the one-way valve and the stopper, and a body member having a first end accepting a first end of the cartridge member and a second end opposite the first end of the body member; providing a cap unit, the cap unit comprising a wash chamber, a one-way valve, and a filter, wherein the filter is either between the wash chamber and the one-way valve or the one-way valve is between the filter and the wash chamber; disposing a sample to be analyzed on the sampling port; disposing the cap unit on the second end of the body member; actuating the cartridge member relative to the body member in a first direction to mix the sample with a liquid disposed in the fluid chamber of the cartridge member; actuating the cartridge member relative to the body member in a second direction, which is opposite the first direction, to extract an analyte from the sample if present, filter the sample, and deliver the sample to an analyzer to sample and process the liquid suspension.

The system provides an integrated, portable, low-cost device for sampling that provides for reduced risk of contamination. The system is hand-operated and can be used without electrical power. The system can operate using small volumes, e.g., less than 100 μL, and can quantify a captured analyte. The integrated nature of the system avoids pre-processing of the sample.

### BRIEF DESCRIPTION OF DRAWINGS

The above and other advantages and features of this disclosure will become more apparent by describing in further detail exemplary embodiments thereof with reference to the accompanying drawings, in which:

FIG. 1A is an embodiment of an integrated system for sampling and processing a liquid suspension; FIG. 1B is another embodiment of the system;

FIG. 2 is an exploded view of the system of FIG. 1, showing the sponge between the filter and the reagent storage compartment;

FIG. 3 is a cross-sectional view of the system in a first position;

FIG. 4 is a cross sectional view of the system when the plunger is midway through a down stroke;

FIG. 5 shows a cross sectional view of the system when the plunger is fully extended, e.g., at a bottom of the down stroke;

FIG. 6 shows a cross sectional view of the system after the plunger has been depressed following the down stroke;

FIG. 7 shows an embodiment of a process for sampling and analyzing a sample;

FIG. 8 shows an embodiment of a process for sampling and analyzing a sample;

FIG. 9 shows an embodiment of a process for sampling and analyzing a sample;

FIG. 10 shows show an embodiment of a process for sampling and analyzing a sample;

FIG. 11 shows an embodiment of a process for sampling and analyzing a sample;

FIG. 12 shows an embodiment of a process for sampling and analyzing a sample;

FIG. 13A is an embodiment of a process of analysis; FIG. 13B is another embodiment of a process of analysis;

FIG. 14 is an image of an embodiment used in an example; and

FIGS. 15A and 15B are illustrations of the methods of patterning and detecting antibodies from the example;

FIGS. 16A to 16C are fluorescent images validating the capture of antibodies from the example;

FIGS. 17A to 17C are flow cytometry graphs of forward scatter versus side scatter, showing the results from the Example;

FIG. 18A is an illustration comparing the steps used in the centrifugation process with the steps used in the syringe-based filtration process, according to the example; and FIG. 18B is a graph showing the results from the Example.

### DESCRIPTION OF EMBODIMENTS

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

Disclosed is an integrated, hand-operated liquid sampling and processing system. The system is capable of accepting an unprocessed liquid suspension sample, processing the sample with at least one fluid stored on the device, and then passing the sample through a separation device, such as a filter, and transmitting the filtered liquid for use in additional operations. An example application is conducting a point-of-care (POC) immunoassay from a whole blood sample.

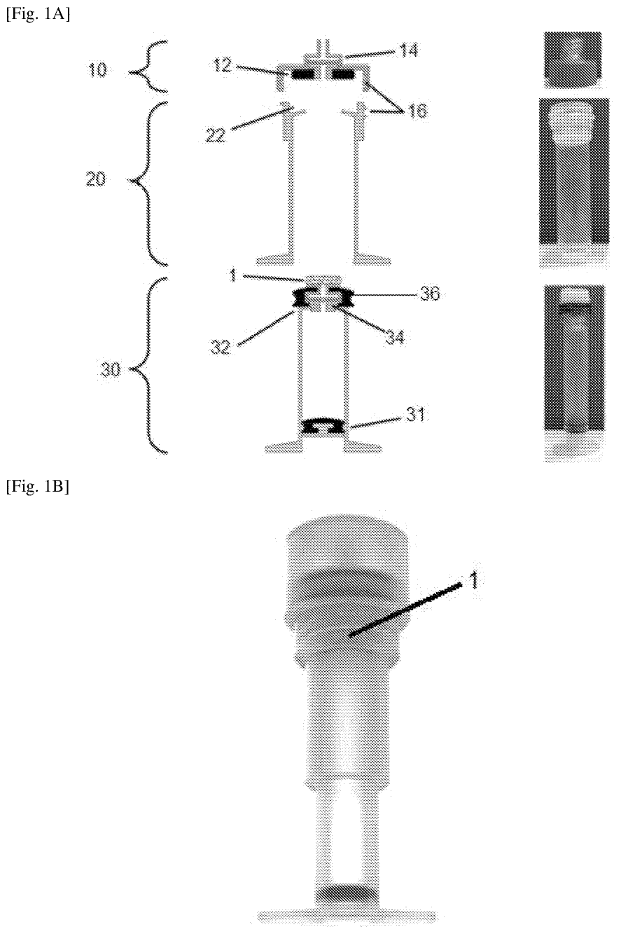

In further detail, as shown in FIGS. 1A and 1B, the system comprises a syringe-like device, with a location for accepting a liquid suspension sample. The system comprises a cap unit 10, a body member 20, and a cartridge member 3. The body member 20 has a first end configured to accept a first end of the cartridge member 3 and a second end opposite the first end of the body member is configured to accept a first end of the cap unit 10.

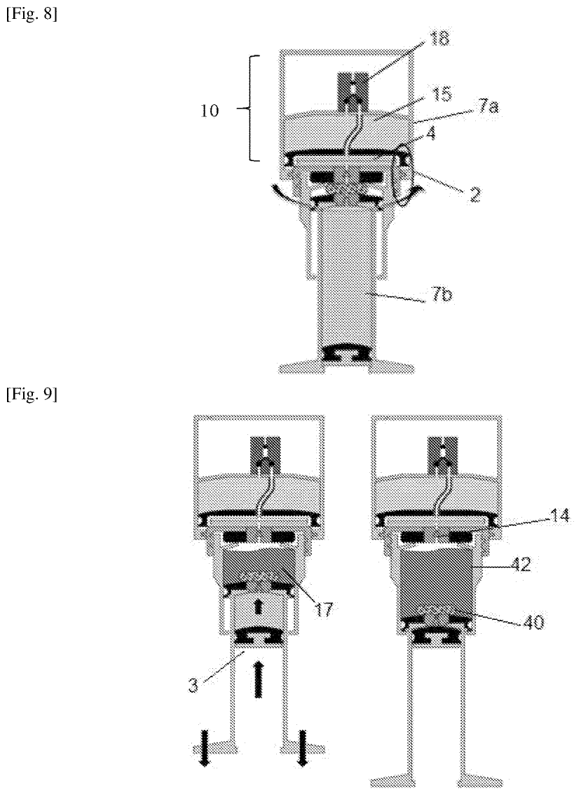

The cap unit 10 is disposed on the second end of the body member 20 and comprises a first gasket 12 and a first one-way valve 14. As shown in FIG. 8, the cap unit also comprises a first compartment 7a (e.g., a wash chamber) and a filter 4 between the second end of the body member and the first compartment 7a.

The body member 20 comprises a gasket seat 22, which accepts the first gasket 12 when the cap unit and the body member 20 are connected. Threaded connectors 16 in the cap unit 10 and the body member 20, are used to connect the cap unit 10 to the body member 20.

The cartridge member 3 comprises a sampling port 40, a second gasket 32, a second one-way valve 34, and a plug 36 on a first end, and a stopper 31 on a second end. The second one-way valve 34 is adjacent to, and in communication with, the sampling port 40. The cartridge member 3 further comprises a second compartment 7b (e.g., fluid chamber) between the second one-way valve 34 and the stopper 31. The cartridge member 3 can be, for example, a plunger.

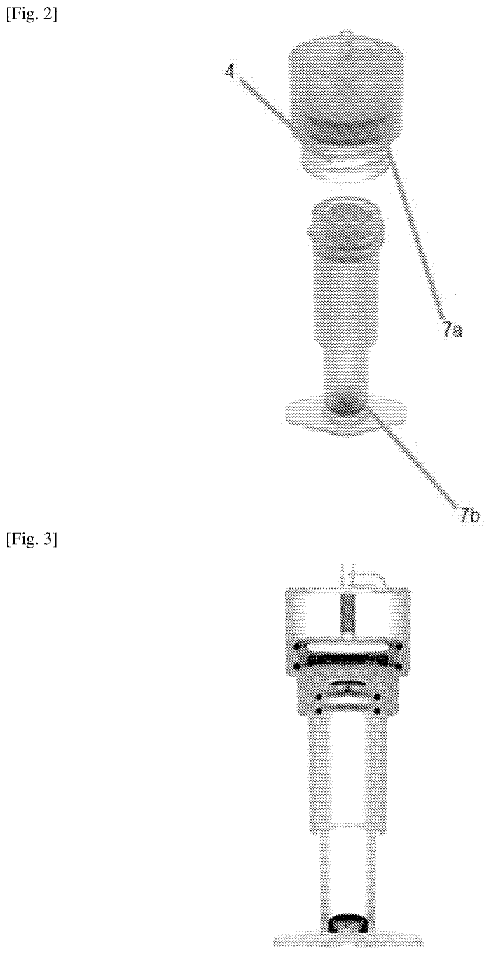

As shown in FIG. 2, the system can form a seal in order to prevent contamination during processing. The system comprises a series of compartments, some of which can contain a liquid reagent for use during sample processing. As shown in FIG. 2, the system includes a first compartment 7a and a second compartment 7b, and a filter 4 disposed between the first and second compartments 7a, 7b.

FIG. 3 is a cross-sectional view of the system in a first position, e.g., an initial position suitable for accepting a sample. In use, pulling down on the cartridge member (e.g., a plunger) causes a liquid to transfer from a storage compartment, through a one-way valve, into a first chamber while mixing with and diluting the sample. Depressing the cartridge member passes the diluted sample through a filter 4, e.g., a membrane.

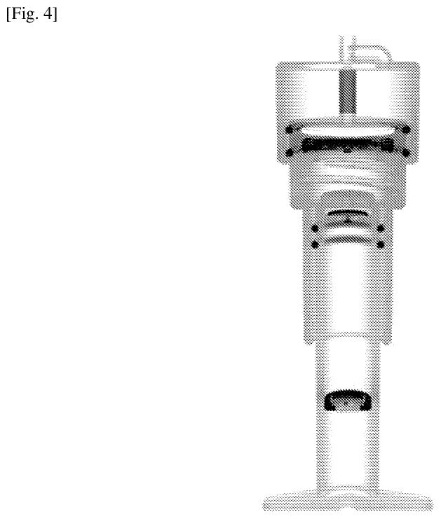

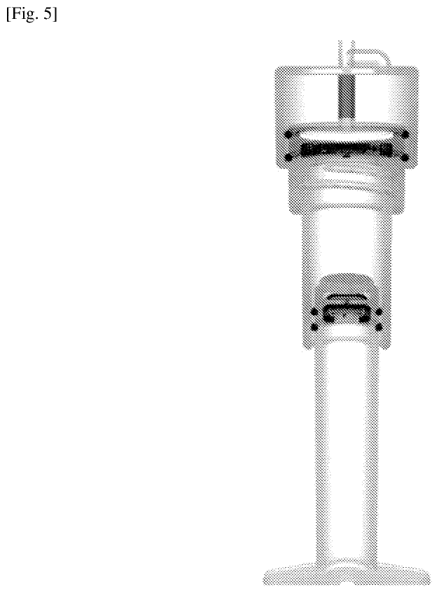

FIG. 4 shows a cross sectional view of the system when the cartridge member is midway through a down stroke. FIG. 5 shows a cross sectional view of the system when the cartridge member is fully extended, e.g., at a bottom of the down stroke. The down stroke of the cartridge member causes a liquid to transfer from a storage compartment, through a one-way valve, into a first chamber while mixing with and diluting the sample.

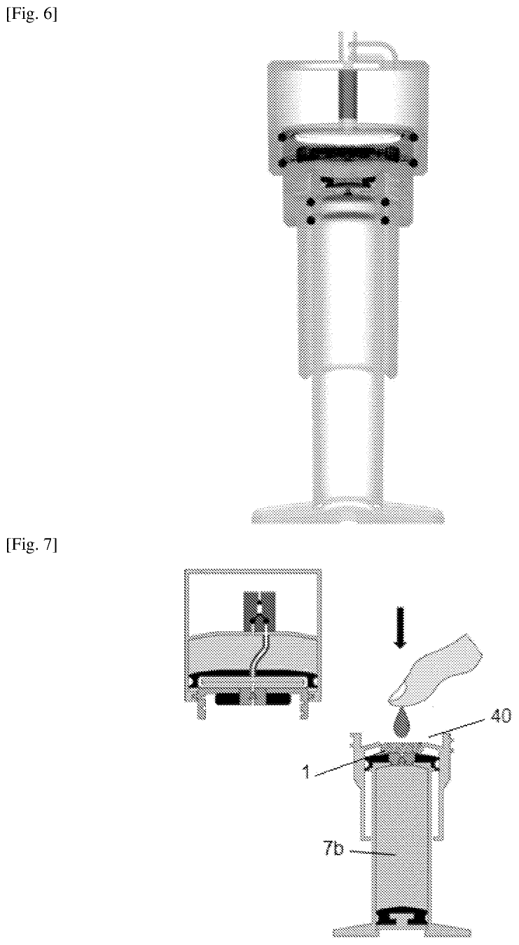

FIG. 6 shows a cross sectional view of the system after the plunger has been depressed following the down stroke. Depressing the plunger causes the diluted sample to pass through the filter 4 and through at least one one-way valve.

In further detail, as shown in FIG. 7, the system comprises a sampling port 40 for collection of a liquid suspension, e.g., whole blood, onto an absorbent 1, e.g., a sponge. As shown, a buffer may be provided in a second compartment 7b.

As shown in FIG. 8, the system may comprise a locking assembly 2 that interfaces with the second compartment 7b, e.g., secondary container or fluid chamber storing a buffer or other liquid reagent. The volume of the second compartment 7b may be any suitable volume, e.g., less than 2 milliliters (mL).

As shown in FIG. 9, actuating the cartridge member 3 causes the fluid to be urged through the absorbent. The cartridge member 3 is freely moving and draws fluid from one compartment into another compartment when moved in a specific direction. As shown by the arrows in FIG. 9, the cartridge member 3 moves the fluid (e.g., buffer) through the absorbent 1 and into a mixing chamber 42 between the sampling port 40 at the first end of the cartridge member and the first one-way valve 14 of the cap unit to provide a sample and buffer mixture 17.

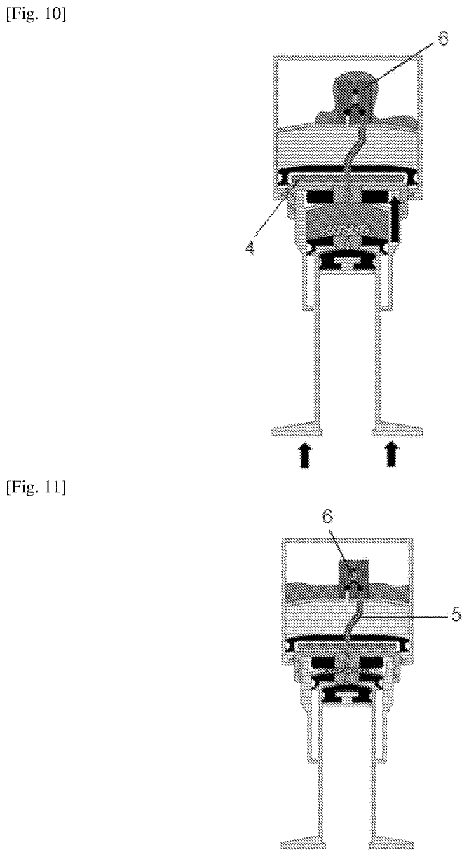

As shown in FIG. 10, further actuation of the cartridge member 3 forces the sample and buffer mixture 17 through a separation filter 4, yielding only a liquid phase.

As shown in FIG. 11, the system can comprise a valved tube 5 or other fluid carrying channel in fluid communication with the chamber holding the filtered liquid to allow extraction of the filtered liquid in a controlled manner for further analysis. Also, in another embodiment, an assay device 6 (e.g., a microfluidic device) in fluid communication with the filter, and filtered liquid compartment, is capable of capturing or detecting a molecular species potentially contained in the sample. When the cartridge member 3 is depressed, the filtered liquid travels from the filter 4 and through the valved tube 5 to the assay device 6.

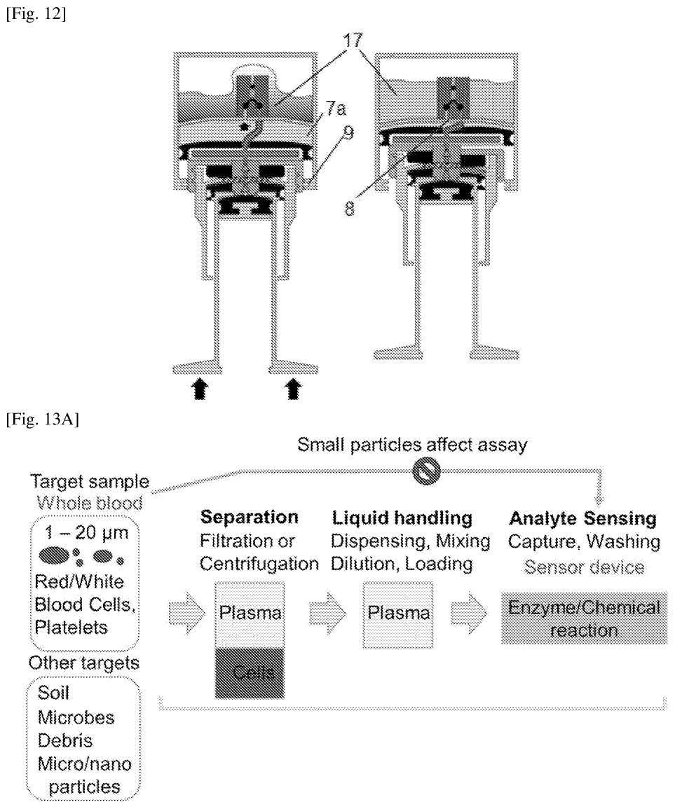

As shown in FIG. 12, the first compartment 7a, which can have a volume of less than <5 mL, can be integrated into the cap unit 10 and holds the wash buffer for the assay. The first storage compartment 7a can be activated by a separate locking mechanism 9. The wash buffer in the first storage compartment 7a is in fluid communication with the assay device 6 and is separate from the filtered sample via a communication path 8. When the plunger is depressed, the wash buffer is urged through the assay device 6 via the communication path 8, after the sample has passed through the same device.

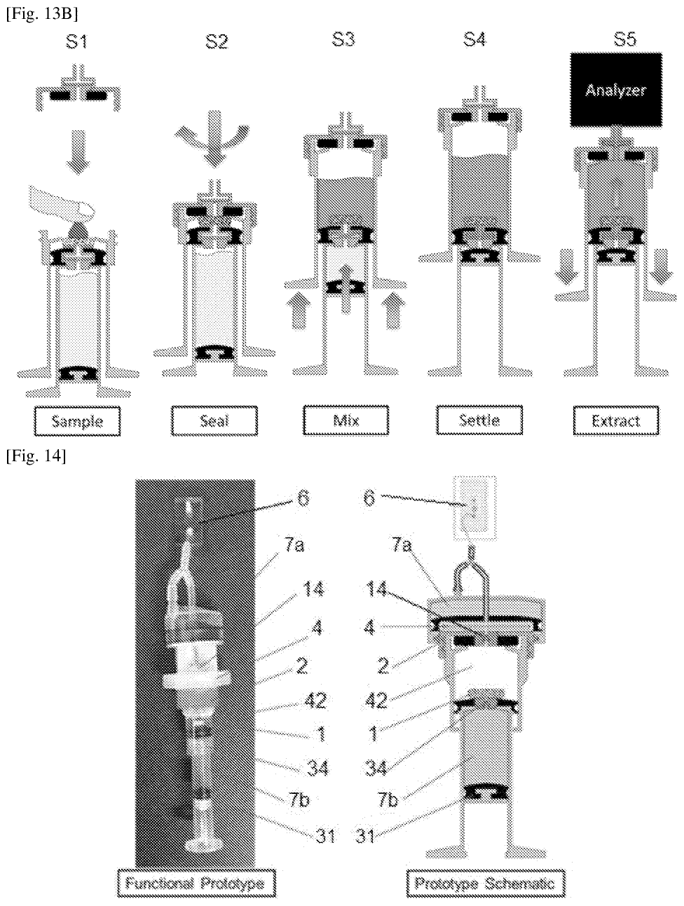

Small particles in a sample can adversely affect an assay, and thus their removal from a sample prior to analysis can be desirable. However, as shown in FIG. 13A, the separation of small particles from the sample can involve multiple separate steps which are time consuming and/or labor intensive.

A method of sampling and processing a liquid suspension comprises providing a cartridge member comprising a sampling port on a first end, a second one-way valve in communication with the sampling port, a stopper at a second end, and a fluid chamber between the second one-way valve and the stopper, providing a body member having a first end accepting a first end of the cartridge member and a second end opposite the first end of the body member, and providing a cap unit comprising a wash chamber, a first one-way valve, and a filter. The filter is either between the wash chamber and the first one-way valve or the first one-way valve is between the filter and the wash chamber.

A sample to be analyzed is disposed on the sampling port and the cap unit is disposed on the second end of the body member. The cartridge member is actuated relative to the body member in a first direction to mix the sample with a liquid disposed in the fluid chamber of the cartridge member. The cartridge member is also actuated relative to the body member in a second direction, which is opposite the first direction, to extract an analyte from the sample if present, filter the sample, and deliver the sample to an analyzer to sample and process the liquid suspension. The analyzer can be an assay device, e.g., a microfluidic device.

An embodiment of a method of analysis is shown in FIG. 13B. The sample is disposed on the sampling port and is absorbed to the absorbent material (S1). The cartridge member, e.g., plunger, when withdrawn, draws the sample into a buffer chamber where it can mix with a buffer (S2). When the cartridge member is depressed the sample is forced through a filter, yielding a liquid phase without solids (S3, S4). An optional fluidic device in fluid communication with the liquid phase can capture a species of interest if present in the liquid (S5). If desired, a wash buffer can be urged through the fluidic device to flush the fluidic device, for example.

The system provides numerous advantages. For example, the system is hand-operated and can operate in the absence of electrical power. Also the system is portable, facilitating its use in a clinical environment. In addition the syringe-like configuration is familiar to clinicians, facilitating adoption by users. Also:
(1) a small volume (<100 μL) of liquid suspension can be processed with any suitable reagents,
(2) the liquid phase can be separated from the solid phase,
(3) the system is integrated, providing capture, processing, and in some configurations analysis of the target analyte in an integrated assay device,
(4) an integrated wash buffer allows for quantification of the captured analyte, if present,
(5) pre-processing of the sample is optional,
(6) sample processing is unsophisticated for the user,
(7) opportunities for contamination are reduced,
(8) the integrated system can capture a target analyte from the sample,
(9) the system integrates a wash buffer to allow quantitative measurements of captured molecular species,
(10) electrical power is not needed, hand operation is suitable,
(11) the system is portable and compact, allowing for transport of large quantities,
(12) the system can be disposable, and
(13) bio-contamination is reduced.

Set forth below are some embodiments of the integrated system and for a method of sampling and processing a liquid suspension using the integrated system.

Embodiment 1: an integrated system for sampling and processing a liquid suspension, the system comprising: a cartridge member comprising a sampling port on a first end of the cartridge member, a second one-way valve in communication with the sampling port, a stopper at a second end of the cartridge member, and a fluid chamber between the second one-way valve and the stopper; a body member having a first end configured to accept a first end of the cartridge member and a second end opposite the first end of the body member; and a cap unit disposed on the second end of the body member, the cap unit comprising a wash chamber, wherein the cap unit comprises a first one-way valve between the second end of the body member and the wash chamber and a filter between the first one-way valve and the wash chamber or a filter between the second end of the body member and the wash chamber and the first one-way valve between the filter and the wash chamber.

Embodiment 2: the system of embodiment 1, further comprising an absorbent on the sampling port.

Embodiment 3: the system of any of embodiments 1 or 2, further comprising a microfluidic device in fluid communication with the filter.

Embodiment 4: the system of any of embodiments 1-3, wherein the body member comprises a mixing chamber between the sampling port of the cartridge member and the first one-way valve of the cap unit.

Embodiment 5: A method of sampling and processing a liquid suspension, the method comprising: providing a cartridge member comprising a sampling port on a first end of the cartridge member, a second one-way valve in communication with the sampling port, a stopper at a second end of the cartridge member, and a fluid chamber between the one-way valve and the stopper, and a body member having a first end accepting a first end of the cartridge member and a second end opposite the first end of the body member; providing a cap unit, the cap unit comprising a wash chamber, a first one-way valve, and a filter, wherein the filter is either between the wash chamber and the first one-way valve or the first one-way valve is between the filter and the wash chamber; disposing a sample to be analyzed on the sampling port;

Embodiment 6: the method of embodiment 5, wherein the analyzer is a microfluidic device.

### Example

A fully functional prototype was fabricated and a microfluidic immunoassay conducted. Shown in FIG. 14 is an image as well as a schematic of the functional prototype with the key components labeled. The processing sequence is identical to that described above. The buffer fluid and wash buffer chambers were pre-loaded with 2 mL and 1.5 mL of phosphate buffered saline (PBS), respectively.

A microfluidic device was patterned with green fluorescent antibodies using a previously described method (Sathish, et al., Analyst, 2017). In this example, antibodies are premixed into the blood sample in order to use the antibodies as a target antigen. Specifically, a 50 μL droplet of mouse blood premixed with red antibodies (1 μg/mL) specific to the immobilized antibodies was loaded into the sample port of the functional prototype.

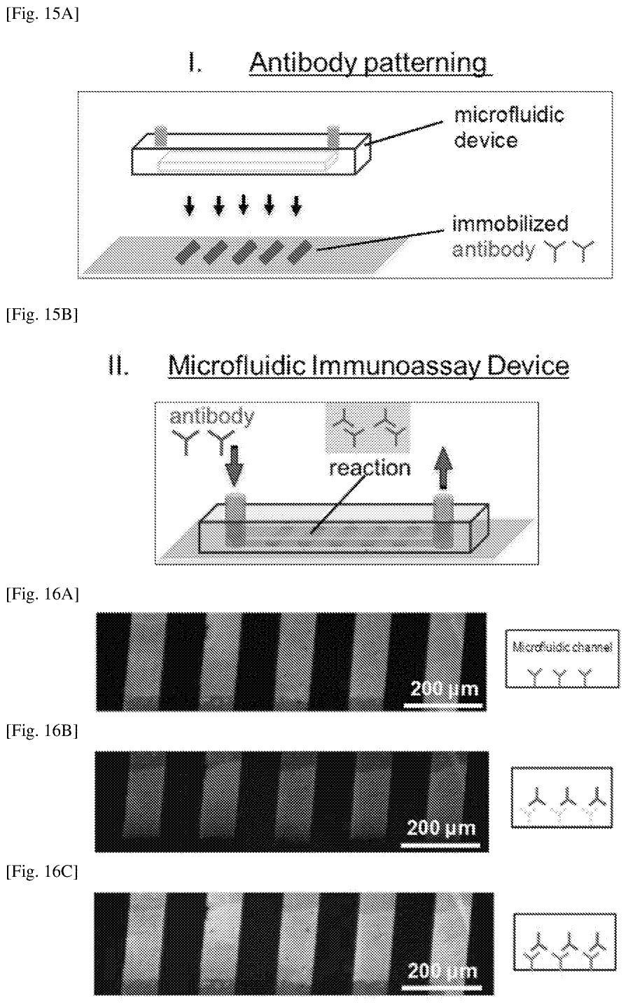

The sample was then processed and introduced into the patterned microfluidic device by the method described above, i.e., by actuating the plunger. The results are shown in FIGS. 15-17. As shown in FIG. 15A, a microfluidic device was patterned with green fluorescent antibodies using a previously described method (Sathish, et al., Analyst, 2017). As illustrated in FIG. 15B, a 50 μL droplet of mouse blood premixed with red antibodies (1 μg/ml) specific to the immobilized antibodies was loaded into the sample port of the functional prototype. The sample was then processed and introduced into the patterned microfluidic device as described in FIGS. 7-9. As shown in FIGS. 16A to 16C, the fluorescence images validate the capture of the antibodies in the postprocessed sample by the immobilized antibodies patterned within the microfluidic immunoassay device. FIG. 16A shows the fluorescent image of the capture antibody (immobilized antibody), FIG. 16B shows the fluorescent image of the detected antibody in the sample (captured antibody), and FIG. 16C shows the merged images of FIGS. 16A and 16B.

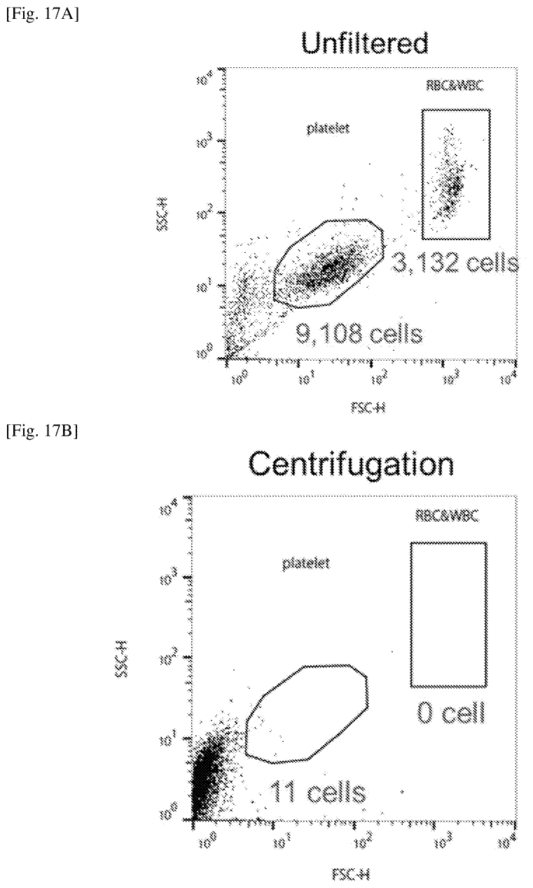

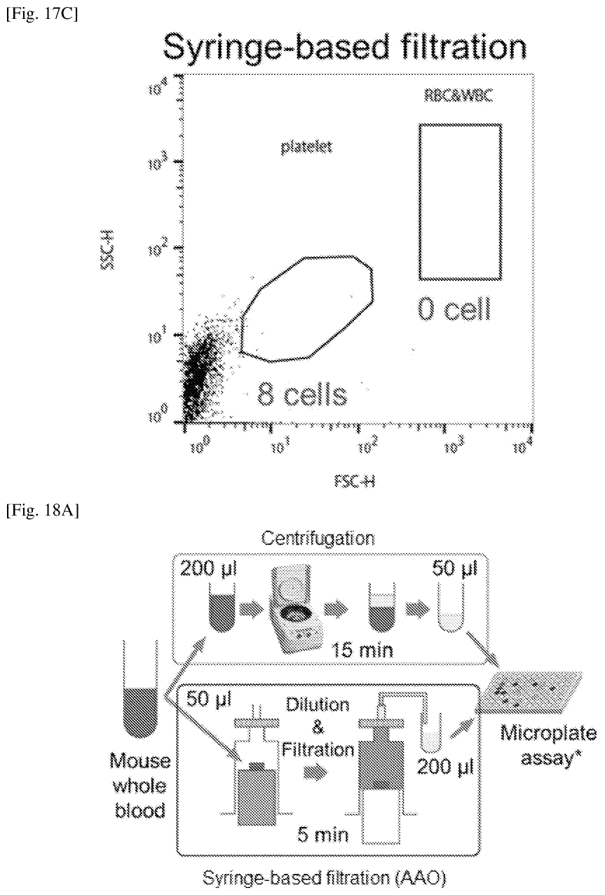

The separation of plasma from a blood sample was evaluated by flow cytometry and the results are shown in FIGS. 17A to 17C. The results show the number of particles (cells) detected in an unfiltered blood sample (FIG. 17A), a sample subjected to centrifugation (FIG. 17B), and a sample processed using the functional prototype (syringe-based filtration system). The functional prototype was equally as effective at separating the plasma from the blood sample as centrifugation, and in a much shorter period of time.

These results indicate that:
i. a sample containing antibodies can be processed and delivered by the system
ii. delivered antibodies can be captured in a microfluidic immunoassay device
iii. the immunoassay device can be washed free of unbound antibodies to enable detection of the immunoassay reaction
iv. the entire process was accomplished in less than 5 minutes

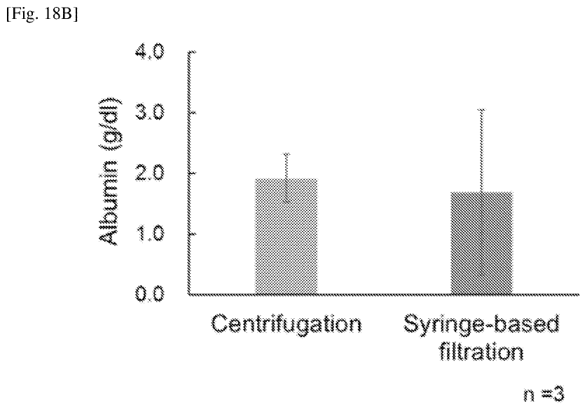

Additional results are shown in FIGS. 18A and 18B. Table 1 below summarizes the ability of the functional prototype system (syringe-based filtration), compared to the standard protocol of centrifugation, to separate mouse blood plasma from mouse whole blood. The system used in this Example included a 200 nm anodized aluminum oxide (AAO) filter membrane, which was found to be suitable for this separation. However, other filter membranes are possible to use.

TABLE 1

Cell removal ratio (%)*

| Plasma separation method | RBC/WBC** | Platelet |
|---|---|---|
| Centrifugation*** | 100.00 ± 0.00 | 99.81 ± 0.10 |
| Syringe-based filtration**** | 99.99 ± 0.02 | 99.91 ± 0.03 |

*Mean ± S.D. (n = 3)
**Red blood cell/White blood cell
***2,000 × g, 15 min
****AAO membrane (200 nm, Anotop ™, GE Healthcare)

As shown in FIG. 18B, the albumin concentration for each mouse blood sample set was determined by colorimetric assay. Albumin is used here to look for “loss” of analyte during the separation steps. The syringe-based filtration of the functional prototype and centrifugation samples show comparable levels of albumin.

It will be understood that when an element is referred to as being “on” another element, it can be directly on the other element or intervening elements may be present there between. In contrast, when an element is referred to as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

1. An integrated system for sampling and processing a liquid suspension, the system comprising:
a cartridge member comprising a sampling port on a first end of the cartridge member, a second one-way valve in communication with the sampling port, a stopper at a second end of the cartridge member, and a fluid chamber between the second one-way valve and the stopper;
a body member having a first end configured to accept the first end of the cartridge member and a second end opposite the first end of the body member; and
a cap unit disposed on the second end of the body member, the cap unit comprising a wash chamber,
wherein the cap unit comprises
a first one-way valve between the second end of the body member and the wash chamber and a filter between the first one-way valve and the wash chamber, or
a filter between the second end of the body member and the wash chamber and a first one-way valve between the filter and the wash chamber.

2. The system of claim 1, further comprising an absorbent on the sampling port.

3. The system of claim 1, further comprising a microfluidic device in fluid communication with the filter.

4. The system of claim 1, wherein the body member comprises a mixing chamber between the sampling port of the cartridge member and the first one-way valve of the cap unit.

5. A method of sampling and processing a liquid suspension, the method comprising:
providing
a cartridge member comprising a sampling port on a first end of the cartridge member, a second one-way valve in communication with the sampling port, a stopper at a second end of the cartridge member, and a fluid chamber between the second one-way valve and the stopper, and
a body member having a first end accepting the first end of the cartridge member and a second end opposite the first end of the body member;
providing a cap unit, the cap unit comprising
a wash chamber,
a first one-way valve, and
a filter, wherein the filter is either between the wash chamber and the first one-way valve or the first one-way valve is between the filter and the wash chamber;
disposing a sample to be analyzed on the sampling port;
disposing the cap unit on the second end of the body member;
actuating the cartridge member relative to the body member in a first direction to mix the sample with a liquid disposed in the fluid chamber of the cartridge member; and
actuating the cartridge member relative to the body member in a second direction, which is opposite the first direction, to extract an analyte from the sample if present, filter the sample, and deliver the sample to an analyzer to sample and process the liquid suspension.

6. The method of claim 5, wherein the analyzer is a microfluidic device.
